SEQUENCE LISTING (8 sequences)

SEQ ID NO: 1 - DNA, 1677 bp, Trichoderma reesei
gacaagcgaaggccttactgcaagaacgagtggcagtctacatcttctacagcggacggagagattatcgaactgcttgtatcatagctggaacacacaatgacccctcactcgattgaccgggccgcgaggccttccgtctggagtggccttgctcttctcctctctacagcacacgcaatcgtgatgcctgatggagtgactggaaaagttccaagtctggggtggaactcgtggaatgcctaccactgcgatatcgatgaaagcaagtttctctcggccgccgaagtcattgtgagctctgggcttctcgacgcaggctacaattatgtcaatattgatgactgctggtcgatgaaagacggtcgtgtggacggccatattgcagtcaacacaactcgcttccctgacggcattgatgggctggcgaagaaggtccacgacttgggcttgaagttgggcatttacagcactgctgggactgcaacttgtgctggctaccctgccagtcttggctacgaggacgtcgatgccgctgattttgccgactggggcgtggactatctgaaatatgacaattgcaacgtcccttcagactggcaagatgaatacgtcgcctgcgcccccgacgccgtccaaaccggccccaacggcacctgctcaaccgcccttgagccaaacctcgcccctccgggctacgactggagcacttccaagtcagccgagcgcttcaacgccatgaggaacgccctggcgaagcagagccgcgagatcgtgctcagcctgtgcatctggggagtggccgacgtcttctcctggggcaacgagacaggcatcagctggcgcatgagcggcgatatttcgcccgaatggggctccgtgacgcacatcatcaacatgaattcgttcaagatgaattccgtcggcttctggggccacaacgacgcggatatactcgaggtcggcaacggcaacctgacggctgctgagacgcggacgcactttgcgctgtgggcggccatgaagtcgccgctgctgatcgggacggatcttgctcagttgtcgcaggagaacattgagttgctgaagaataagcatctgctggcgtttaaccaggacagcgtctacggtcagcctgccacgccctataaatggggcgtcaaccctgactggacctttaactatacgaaccctgccgagtactgggccggtccatcgtcaaaggggcatctggtgctgatgatgaacacgctggatcacacggtgagaaaggaggccaagtggtctgagattccggggctgtctgcgggacggtatgaggtccgggatgtgtggacggacaagagccttgggtgcctcagctcgtacaagacggctgttgcagctcatgacaccgctgttattctggttggcaagaagtgccgaaactggtgatggggtcggaagaggatggatatcgactggacctctggttgaatgcttgtcatcgtctgcatcctgacgggagccggaaggactgtggctgctgtctgcttcagatccagatggaagcacccttatccagactatccagaaatcacaagccaattgcaggattccagtcttctcagttactaggaggtgaataagtaaggtagagatggctgacttgtcctgctaaaaaaaaaaaaaaaaaaa

SEQ ID NO: 2 - protein, 444 aa, Trichoderma reesei
MTPHSIDRAARPSVWSGLALLLSTAHAIVMPDGVTGKVPSLGWNSWNAYHCDIDESKFLSAAEVIVSSGLLDAGYNYVNIDDCWSMKDGRVDGHIAVNTTRFPDGIDGLAKKVHDLGLKLGIYSTAGTATCAGYPASLGYEDVDAADFADWGVDYLKYDNCNVPSDWQDEYVACAPDAVQTGPNGTCSTALEPNLAPPGYDWSTSKSAERFNAMRNALAKQSREIVLSLCIWGVADVFSWGNETGISWRMSGDISPEWGSVTHIINMNSFKMNSVGFWGHNDADILEVGNGNLTAAETRTHFALWAAMKSPLLIGTDLAQLSQENIELLKNKHLLAFNQDSVYGQPATPYKWGVNPDWTFNYTNPAEYWAGPSSKGHLVLMMNTLDHTVRKEAKWSEIPGLSAGRYEVRDVWTDKSLGCLSSYKTAVAAHDTAVILVGKKCRNW

SEQ ID NO: 3 - DNA, 2438 bp, Trichoderma reesei
cacagttggccccggactctgtggtggccaatcttcacgatgctcggcgctccctctcctagaaggctggcggacgtcctcgccgtgaccgcgggactggtggcctctgttagggcagcaagtcccatctccgtgtctggcaagtcgtttgccctcaacggcgacaacgtctcgtaccgcttccacgtcgacgacgactctaaggacctcatcggcgaccactttggcggccctgccacggaagatggcgtcttcccccccatcatcggccccatccagggctgggtcgacctcatcggccggcagcggcgcgagttccccgacctgggccgcggcgactttcgcacgcccgcggtgcacatccggcaggcggcgggctacacggtcagcgacttccagtacaagtcgcaccgcgtcgtcgagggcaagccggcgctgcgcggcctgccgtcgacgtttggcgacgccggcgatgtgtcgactctggtcgtgcacatgtatgataactacagctccgtggccgccgacctgacctactccatcttccccaaatacgacgccattgtgcgcagcgtcaacatcaccaacatgggcaagggtaacatcaccattgagaagctcgccagcttgagcgtcgatctgccgtatgaggactttgacatgctggagctcaagggtgactgggctcgcgagggaaagcggctgcgtcgcaaggttgactacggctctcagggtttcgggagcacgactggctattcttcccatctccacaaccccttcttctcgctcatcacgcctacgacgaccgagtcccaaggagaggcatggggcttctcccttgtgtacactggctccttctccgtcgaggtcgaaaagggttcgcagggtctcacgcgagccgccattggcgtcaacccctatcaactgtcgtggccgttgggccctggcgagaccttcagcagccccgaggcggttgccgtcttctctaccactggcgttggcggaatgtcgcgaaagttccacaacctgtaccgcaagcatctgatcaagagcaaattcgcgacgcagatgcaccccgtcttgctcaacagctgggagggcctcggcttcgactacaacgacaccaccattctgcatctggcgcaggagtctgccgatctcggcatcaagctgtttgtgctggatgatggctggtttggcgtcaagcatcctcgagttagtgacaatgctggcctgggcgactgggaggcgaaccccaagaggttcccgcagggcctgccagacttcattagcgacgtgacaaagctcaaggtggccaactcctctgatcatctgcagtttggcctctggttcgagcctgaaatggtcaaccccaactcaaccctatacatggaacacccggactgggcgattcacgccgggtcgtaccctcgtaccttgacgaggaaccagctggtgctcaacgtcgctctcccagaggtgcaggatttcatcattgagtcgctgtccaacattctgagcaacgccagcatttcgtacgtcaagtgggacaacaaccgcggcatccacgaggccccttaccccgggctcgactacgcctacatgctgggcctgtaccgcgtctttgacacgctgtcgtcaaagttccccaatgttcgctgggagggttgcgcgtctggcggcggccgcttcgatcccggcgtgctgcagtactttcctcacatctggacgtctgacgacacggatgccgtggagcgcattgcgatccagtttggcacgtcgctcgtgtatccgccgtcggccatgggagcccacgtctctgccgtaccgaacggccagacgcagcgcacgacgtcgattgccttccgcgcccacgttgccatgatgggaggttcgtttggcttcgagctcacccctgcggagatgccggaggacgacaaggcgcagatcccgggcatcattgcgctggcggaaaaggtgaaccccattgttgtcaagggcgacatgtggcggctgagcctgccggaggagtccaactggccggcggcgctcttcatctcgcaggacggcagccaggcggtgctgttctacttccagatccgggccaacatcaacaacgcgtggccggtgctgcggctgcagggcctggatgcgtcggccaagtacaagattgacggcaaccagacgttctcgggggcgacgctgatgaacatcgggctgcagtatcagttcaatggggactatgacagcaaggtggtgtttttggagaagcagacatgagctgattttgtgaacccatcaccatgacctagatgacctacaggctttgtttgatgcatcgtttcttcagtagcacttatgtatggtagcttagcattcagtagttaatacacagcaggcgttttctgactgaaaaaagaaaaaaaaaaaaaaaaaaa

SEQ ID NO: 4 - protein, 746 aa, Trichoderma reesei
MLGAPSPRRLADVLAVTAGLVASVRAASPISVSGKSFALNGDNVSYRFHVDDDSKDLIGDHFGGPATEDGVFPPIIGPIQGWVDLIGRQRREFPDLGRGDFRTPAVHIRQAAGYTVSDFQYKSHRVVEGKPALRGLPSTFGDAGDVSTLVVHMYDNYSSVAADLTYSIFPKYDAIVRSVNITNMGKGNITIEKLASLSVDLPYEDFDMLELKGDWAREGKRLRRKVDYGSQGFGSTTGYSSHLHNPFFSLITPTTTESQGEAWGFSLVYTGSFSVEVEKGSQGLTRAAIGVNPYQLSWPLGPGETFSSPEAVAVFSTTGVGGMSRKFHNLYRKHLIKSKFATQMHPVLLNSWEGLGFDYNDTTILHLAQESADLGIKLFVLDDGWFGVKHPRVSDNAGLGDWEANPKRFPQGLPDFISDVTKLKVANSSDHLQFGLWFEPEMVNPNSTLYMEHPDWAIHAGSYPRTLTRNQLVLNVALPEVQDFIIESLSNILSNASISYVKWDNNRGIHEAPYPGLDYAYMLGLYRVFDTLSSKFPNVRWEGCASGGGRFDPGVLQYFPHIWTSDDTDAVERIAIQFGTSLVYPPSAMGAHVSAVPNGQTQRTTSIAFRAHVAMMGGSFGFELTPAEMPEDDKAQIPGIIALAEKVNPIVVKGDMWRLSLPEESNWPAALFISQDGSQAVLFYFQIRANINNAWPVLRLQGLDASAKYKIDGNQTFSGATLMNIGLQYQFNGDYDSKVVFLEKQT

SEQ ID NO: 5 - DNA, 2004 bp, Trichoderma reesei
gacaagccaaaggcgcaaagcgttcagttacatcgcagcacaatgtcgcccagtgctgcagttctcattcccctcgcagcggcagttctgcttcgtcctgtggtcggtcaaacgcaatgcggcggcaatctgtacactccggggacgctcaacttcactctggagtgctacaatgcgtttcaggactgcgtcgctcagtttgaggccaacgcaagccaagtcgactgcaacgacggcaagggaaacctgttcatgcaacaacaggccaacttgggggcctcgccaggaagccagaacaacgacgccatcattgcctttcaggacattcgcgatctctgtctgctcagcggttcaacaactgcaacgtggggatatagcgacaaccagtggtattgggcggctgccgaagatgcctgctacacaaacgatcccacgaggaccgacgttgtcaagactcacccggcgccgttttgcatccagaaccgcgactcttcactgcctgagtgctacccacagccggatgccacccctcccggcggcccactcaaggtcatcaagacggccaagacgcgaaacgggttcaagtcctcagcccgaggctggaatacctacggcgtccaagctctggtcaacggttcccaggtcgtgccgtcctttgctggacagtcgggtctgttctacacccagaagttcgtcgagactcagtgtggagttcttgcccgacccgagttcaagaaggctgggtacgatctctgcagccttgattcgggttggcaggctactaccgccgttgatcagcatggtcgaatcatctacaacaccacgcgattcaacctccccgagcttgcttcatggctacacaagagggatttgaagctcggcgtatacattacccctggcgtgccatgtctggctcacaaccaaaccatcctcggcaccaacatcaagatcaaggatgtcttgaatgggaacaacgatcagatcaactgtgactttgacttccgcaaagatggtgtccagcagtggcacgattccgtcgtcgcacaatgggcttcctggggcgtggacatgctcaaactggactttctgacgcctggctccccttccaatggcgcaaacctcgcgtgtgacagttcagatgctgttcgagcataccagaaggcaatcaagaagtcaggacgaaaaattcgcctcgacatctcgtggaagctttgccgcaacgaaacctggctacctatctggagcgaccttgctgagtcaatgcgcactgatcaggatctcgacaactacggcaccaacactttgatggcatggcaggtcggccagcgcgcgattgagaactacaggcagtacatcggtttgcaagcgcagagaaatgtccccctcacgatctatcctgatatggatgctcttttcacggtcaaccccgagcatctcgccggtgtaaacgacactattcgctatacggttcagaaccactggcttggagctggcgccaacctcatcattggtggcgatatggagcaggtcgatgctctggggctcaagctgactaccagcaagcaatcgattgatgcggcagacttttttgcaaagtatcccatgcagcctcgtaaccccggaaccggaagcaacgccgccaagcagctccaggcctggatcggtggcccttcggatgaccacgaggcttatgtgctcattgtcaactacgggccagacttggggaatggtggcttttcaaccaagctgtatggaaagcagaaagtgacagtgtcgttgaaggatcttggtatctctggctccgcctggacttttaccgacatatggtccggcaagtccagcagagtgactgggtcctactctgcctggctcaccgagggcgagtcccagcttctgcgcctgaaaaggactcactagtgaacacgaagacgtatagaggtttctagagtttagcgaagcaatacactttataatccctaaaaaaaaaaaaaaaaaaaaaaaaaa

SEQ ID NO: 6 - protein, 624 aa, Trichoderma reesei
MSPSAAVLIPLAAAVLLRPVVGQTQCGGNLYTPGTLNFTLECYNAFQDCVAQFEANASQVDCNDGKGNLFMQQQANLGASPGSQNNDAIIAFQDIRDLCLLSGSTTATWGYSDNQWYWAAAEDACYTNDPTRTDVVKTHPAPFCIQNRDSSLPECYPQPDATPPGGPLKVIKTAKTRNGFKSSARGWNTYGVQALVNGSQVVPSFAGQSGLFYTQKFVETQCGVLARPEFKKAGYDLCSLDSGWQATTAVDQHGRIIYNTTRFNLPELASWLHKRDLKLGVYITPGVPCLAHNQTILGTNIKIKDVLNGNNDQINCDFDFRKDGVQQWHDSVVAQWASWGVDMLKLDFLTPGSPSNGANLACDSSDAVRAYQKAIKKSGRKIRLDISWKLCRNETWLPIWSDLAESMRTDQDLDNYGTNTLMAWQVGQRAIENYRQYIGLQAQRNVPLTIYPDMDALFTVNPEHLAGVNDTIRYTVQNHWLGAGANLIIGGDMEQVDALGLKLTTSKQSIDAADFFAKYPMQPRNPGTGSNAAKQLQAWIGGPSDDHEAYVLIVNYGPDLGNGGFSTKLYGKQKVTVSLKDLGISGSAWTFTDIWSGKSSRVTGSYSAWLTEGESQLLRLKRTH

SEQ ID NO: 7 - DNA, 8777 bp, Clostridium cellulolyticum
ctattgatgcactagattgtgccgttttcaagaaacttctgttaggtattatttaaaaattacccgggtagaactatggtattttagtccatatatggtttaaattgttatatctgaatttgtaaattggttaaggaggcaaacatgttaaaaactatgagtatacttctaccatgcttattgatattttcacttatattcagtgtacaaatacctttatcagcttcagcagcaaatgttgaactcctaaagcagttcgacatggaacaggtaaaaataacagatacatattatgtaaatgcacttaataaagaggttgcctacttgcaggcaattgatccaaaccgtttgttggtgggttttaagaaaacagctggcttatcaacaacttatagctattatggagggtgggaaaacaataccctgattcaaggccataccatgggacattacatgtcggcacttgctcaggcttataaaaacactaagtccgacccgacagtaaatgcagatttgaaaagccgtatcgatttgattatatccgaattgcaggcttgtcagaataaaaacggcaatggatatttgtttgcaactccggctacccaatttgatgttgttgaaggaaaggcgtccggttcaagctgggtaccgtggtataccatgcacaaaatcatgtccggtcttcttgacatttataaatttggaggcaaccaaaccgcattgacaatagcaaccaacttgggaaattggatttacaaaagagtaaacgcttgggattctgcaacacagtcaagggtattgggtgttgagtatggaggaatgaatgactgtctctatgaattgtataagctgactggtaatggcaaccatttaacagcagcacataaatttgacgaaaattcactatttaacaccatcgctgcaggcacaaacgttttacccggaaaacatgccaatacaactatcccgaaattcatcggtgctttgaatcgctacagcactctaggaacatcagaatcatcatacttaaaagcggcacagcagttctgggccatagttttgaaagaccatacatatgtaacagggggcaacagcgaagatgagcgtttcagggacgctggcaaactggatgcatacagggataatgtaaataatgaaacttgtaatgtaaataatatgctgaagctgactaaagagctgttcaaggcaacgggcgacgttaaatatgcagattactatgagaatgcattgataaacgaaatcatggcttcacagaatccggaaaccgggatggctacgtacttcaaggctatgggaactggatatttcaaggtattcagttcccaattcaatcatttctggtgctgtacgggaacgggaatggagaatttcacaaagctgaatgacagcctgtattataataatggttccgacctgtatgtaaacatgtatctgagttctaccctgaactggagcgaaaagggtctttcactgacacagcaggccaatctgccattatcagataaagtaacctttactatcaacagtgcttcttcatcagaagtgaaaattaaattcaggtcaccagcatggattgctgcaggacaaaatattacggtcaaagttaacggtactccaattaatgttgacaaggcgaatggctatcttgacgtcagcagagtgtggcagacaggagatacggttgagttgaccctgcccaccgaagtaagggtatccagactgactgacagccccaatacggtagcctttacatatggtcccgtagtattgagtgcaggtcttggaactgaaagcatgacaactcaatcacacggggtccaggttttaaaagcaacgaaaaatgtgactatcaaagagactattaatattaataccgccgccagtcccagcattgacaattggcttgccaatataaagaacaatttggttcaaacgcctgggaagctggaatttacattgaaaaataccgacgaggataaccatttggtattcacacctcattatcaaagatacaaggacaggtatggtatctacttcaagctgggaacgtatgagggtaaacaacccacggataatttgcttgacaatccggatattgagtcagggaacaccacaggatggactgtgaatggtgcgggtacaattgcctcttcaacagtacaaaagcactcgggaagctatagtctgctgcatacaggcaggacaggagcctggaacgggcctattcagaacattacaacaaaagttcagaatggtaacacgtatacttgttccggctgggtaatactggacaacactgccagtgccccgataacaatgactatcagaaaaacggatgacaacggaacttcctatgtcaatattgccactgctaccggaagcaatagttcctgggttcaattgtcaggtaactataccttaaatgttacaggtgcattgactgacctgagtatatattttgaaggaccggacagcggcaccaatttttatgtggatgatgccttagttaaggtttatggcaaaactaccttctatcagaatacttcttttggcggtactgcggtgtcgctgaatccaggcagctatactactgctcagctcactgctgcaggtatttctgataactgggcatcatcaatcaaaatacctgaaggctatacggttgagatttatgatgatgacaatttcactggtacaaagtggtcttttagtgcagataattcgaactttatagaagccggatgcaatgacaaaatgtcttccgtgaaaattttccccactctgagtcaagtgaagtatggggatattaacagggacggtactgtagatactattgactttgcacttttaaagcagtttttgttgggtgctcaggtcacaattgattcggtagcggctgatttggacggcgatgaatctgtgacggcaatggattttgcggtatttaagaagtatctgctgggacaaataacagagttgcctgctttttgatgttccgatggaattatttgattccaaatttattgatatagcaaatcagccgattcgttctggttttagaacagtctaaataacgaaaatttaggggggataaatagaatgaaaaaagttcgtacagtcagtacaattatctttttaggacttatgattaccgtaattatggttttgaatactggtgcatgggataacggtcttgcaaaaacaccaccgatgggatggaacagttggaacatattccatggagacattaatgaaactaaaatcaaacagattgctgataccatggtaagctcgggtatgaaggaagctggctatgtatacctgaatctggatgataactggatggcaaatcctgcaagagattccaatgggaacttgcgggccgatcctacacgattcccaagtgggattagggctttagctgattatgtacatgcaaaaggtctcaaactagggatatacggatgtcgtggaacaatgacctgtatgaatattcctcaaagcggaagcaagggttatgaggacaaggatgcaaagacatttgcttcatggggaattgattaccttaaatatgataactgcaatatacctaacggaagtgacatgaaaaccgattaccagaaaatgcagaccgctcttgcaaattgcggaagaccaatagtattcagtatatgtgcatggggatatcagagctggatgcctgcaacaggtaatttatggcgtactaccggggatttcgctgataagtgggataacggaaacgaatggttcaaaggtattataaatgcaattgatggtaatgcacaatacacaagttcagccgcacccggtgcatggaatgatcctgatatgcttgaaatcggaaacggtggatgtacaacagaggaataccgtacacagatgagtatgtggagtatgatggcttctccccttattgcaggaaatgatataaggaccatgtcacagacaacaaaggatattctattgaataaggaagtaatagcaatagaccaggatcctgcaggagttcagggaaaaagagttaaaagtgcaaatggtcttgagatttgggtaaaaccactgggtacgaatggtacaactaaggcagttgctttattgaacaggaattcggcaacatccaatattacagttaattggtcagatataggtgtaagtggaagtgttacggtcagggatttgtgggctaaatctgacaaaggcagttttacgggctcatacacagcgtctgttccttcacatggaactgttttgattaagatttccactgagccgccggcacctgttgatgcaacaaagcaaatagaagcagagagttatagcaatcagtcaggaatccagacagaaacctgttcggaaggcggagaggatgtaggctttatcgaaaacggggactatactgtttacagcattgtggatttcggcgatggagtcggaggcttccaggcaagagtagcaagtgcgaccagcggaggcaatattgagattagacttgacagccctgccgggactttaattggaacctgtccggttgccggaacaggggattggcagacttatactgatgtaaaatgtactgtcagcggggcaacaggaaaacatgatgtataccttgtatttaaaggagatagcggatatttattcaaccttaattggttcacatttactccaggaagtgtcaatacgggtacattgggtgatttaaattccgacggacaagtagacgcgatagatttacagttattgaaaaagtatattttaggactgggagcaatcgaaaatacaaaactggcagatttggatgccaacggagatatcaatgcaatagatttttcactgctgaaacaattcttactaggcataaggaccagctttccggggcagggggcagcataatttagcgatatcctgataggaggttgaaaatgaaaagaaagattttatctgttcttttgcttgtcacaatgactacagcattgttttcagctacaccgatgaataccgcttcagccgcaagtacggactttgtactagacggaaacaatatcaaagcgggcaacatcaacggtctcacatttaaggggttcggcgtcctcagtggaaacagctcaagtgcactgttgatggattacaagtcggagcatcctgagaaatatacagaattactgcaaattctgttcggtggaaaaaatccgattatgacacatgtcaaaattgagatgggtaatgaccgtaacaactccaccggaccagatccctcaacaatgcgttgggaaaatgagacggctaatgtcaaaagacaccccggatttcaacttgcggctgatgccaagaaggtgaaccccaatcttaaagtcagcatattacgctggaatgcccccggttgggcaaatagcaatgataagatttatacatggtataagaacaccatattagcagcataccgtcaatatggttatatgattgattacgtaaacccgggggtcaacgaacaaacaccgaatttaacctggactaagcaatacgcccagcgtatcaaaacagacagtacaggttttaacaatgctgaagaacgggcactttacaacaatattaaggtggtgatttccgacgaagtttccgtcggttccttcggggatgatatggtcagtgattccacccttcgtgatgccgtatctgtcgctgcatatcactataatactgatgacaatagcttgggaagtttcaaacagcttgccgaatcctttgacaaagaggtgtggaatagtgaagcacaggccacttttagtaattcgtcctttcgtcccaataacaatatgaaagatccaacagtagcaggaaccggcataggaggcacaaatgggccactggaaatgggaaatactgttataaaggggtttgtaaattcaaggaggacacattttatctatcagccagtcattgggtccttctatgagggtgggcagtactcttttaaggaattggtatccgcacgtgatccttggtccgggtggattcactatgatgccggtcttgtcatacttcggcatttcagttggttttcaaaggctggctgggaaaatgagagtaataccgcaggggtttggagggctgtacctcaggcgagtttcacaggtgcgacgggtacgaatcctgttaatgggcgtaatggcactcccagttatatgacactagcttctccggacaagcatgatttttcaactatcttcattaacgatagtgaatattccaaaacctacacgtttaagaccatcaatatggcttattcgggaaacccctcgctggaagtatgggaaactagagcagctgataagggagcatcttttaatagtaattacatgaagtacactggaacggtttccacaaatagcagcggagtttatacagtaaacgtaaaaccttattcgattgttacagttacaacattaagtaacagtggaaaagcagagttcaatacgcctcttccggtggagggggaccgcccggttctggatacagacaagacaggttcaatgcaggataccagggataatatattatatgctgacaactttgattattccagcaaaactgcccctgtcatagcggaggggggacaaatcacaggaacccaaagttatatagattcccgaggaggctcaaaaagtgctataccacgttatgccagcgacagaaacggtgcatttgaagtgtatcttcctgacgggtccagcaactatatccttcgccaacaggtgaatcagtcaagtatggggcttgggggcacatggaataatggtaatccaatcaccggcattggagataaccgttggatgaactataaggcaagtgtggatgtttcatttgaacacaacagtacagagggcggtaacaattatgctgcaatcggtgccagacagcagggtggtgaaaattcacactacttaaatggtactccttatatactgaaattctggtttgacggcggttggtcgctactagtaaatggaagttccgtggcaaatggtaatgtagcaagcggctcgggtggagtgaaaatcagtggttttaatacagcttataatgcatggcataacatctctatcatggttgcagacaataaggtgactgcgtatctggacaataccatcctttatacctatacagatactacccaaagattgtccgggcgtattgatctggcaagcggctattacaatacttgttttgacaatttgaaggttgaaacaatagacggttatgcaccttactactctgaaatgctggacaatctggaaatgtacgatttgtcttctgtttctgctacaaagcttgtttacggcggttcttgggcacatgaaaacggcaaatccatgtacaattaccaacgatcactttccacgagccagggaataggtgctactattcagtattcattcactggcaccgggctggacattcttggagccaacaacggttctgctaagttagaggtaactgttgatggaagagttgttaattcctcagtgggaaccatggtttcagggaatttacaccaaaactttacgcttcacggtcttgagtacggtaagcatacggtttgtttgaaggtgttaagtggtactatggttgtcgatgctgttggggttgttgcaaacatagccggtgcttcggagatacccgttgaacaatctgcgtattcaaggatagaagcagagagctacagcaaccagtcaggaatccagacagaaacctgttcggaaggcggagaggatgtgggctttgttgaaaacggcgactatactgtttacaacaatgtggatttcggcgatggtgtcggaggcttccaagcaagagtagcaagtgcaaccagtggaggcaatattgagatcaggcttgacagctctaccgggactttgataggaacttgtcctgttgccggaacaggggattggcagacttatactgatgcaaaatgtactgtcagcggggtaacaggaaaacatgatgtataccttgtatttaaaggagatagcggatatttatttaatcttaactggtttacatttagtgagaaaactgtcatagggaatttgggtgatataaactcggacggacaagtagatgcaatagatttacaggtattgaaaaagtatcttttgcaactaggggaaattggagatacgaagctggcagatttggatgccaacggagaaattaacgcaatcgatttttcattactcaaacaatttttactgggtactattattagttttccgggagaagcactataaggggacatcaccgttccatatgtgtgaagaattacaaatataatataaggaggcttttttggtatgttgagaaagaaaattttatgtatatttcttgtgactgtattaatgctaactatattaccaatacctcaacagacggtaatggctgatacaggggtacttaaggaacttaaaggaactgacatctacaacggattaaggggactgaattttaacgagggttggaaattcaataagggggatgtaagcaacggccagagtaccgggtataatgacagcggctggtcaggtgttacactgccacatgactggagtatttataacacttttaataaatcctcagcagcgggtgcaggaggaggttatctggatggaggaatcggatggtacaggaaaacctttaccgtaccttcggattatacaggaaagaaggtattcatt

SEQ ID NO: 8 - protein, 604 aa, Clostridium cellulolyticum
MKKVRTVSTIIFLGLMITVIMVLNTGAWDNGLAKTPPMGWNSWNIFHGDINETKIKQIADTMVSSGMKEAGYVYLNLDDNWMANPARDSNGNLRADPTRFPSGIRALADYVHAKGLKLGIYGCRGTMTCMNIPQSGSKGYEDKDAKTFASWGIDYLKYDNCNIPNGSDMKTDYQKMQTALANCGRPIVFSICAWGYQSWMPATGNLWRTTGDIADKWDNGNEWFKGIINAIDGNAQYTSSAAPGAWNDPDMLEIGNGGCTTEEYRTQMSMWSMMASPLIAGNDIRTMSQTTKDILLNKEVIAIDQDPAGVQGKRVKSANGLEIWVKPLGTNGTTKAVALLNRNSATSNITVNWSDIGVSGSVTVRDLWAKSDKGSFTGSYTASVPSHGTVLIKISTEPPAPVDATKQIEAESYSNQSGIQTETCSEGGEDVGFIENGDYTVYSNVDFGDGVGGFQARVASATSGGNIEIRLDSPAGTLIGTCPVAGTGDWQTYTDVKCTVSGATGKHDVYLVFKGDSGYLFNLNWFTFTPGSVNTGTLGDLNSDGQVDAIDLQLLKKYILGLGAIENTKLADLDANGDINAIDFSLLKQFLLGIRTSFPGQGAA

(19) United States
(12) Patent Application Publication
(10) Pub. No.: US 2015/0273111 A1
(43) Pub. Date: Oct. 1, 2015

(54) MICROBIAL ENZYMES FOR REDUCTION OF ALPHA-GALACTOSE FROM COLLAGEN BASED TISSUE

(71) Applicant: LIFECELL CORPORATION, Branchburg, NJ (US)

(72) Inventors: Rick T. Owens, Stewartsville, NJ (US); Niraj P.E. George, Havertown, PA (US)

(21) Appl. No.: 14/676,351

(22) Filed: Apr. 1, 2015

Related U.S. Application Data
(60) Provisional application No. 61/973,470, filed on Apr. 1, 2014.

(51) Int. Cl. A61L 27/36

(52) U.S. Cl. CPC A61L 27/3687; A61L 27/362; A61L 2430/40

(57) ABSTRACT

Methods for preparing a non-human tissue matrix for transplantation are provided.

### RELATED APPLICATIONS

This application claims the benefit of priority to U.S. Provisional Application No. 61/973,470, filed on Apr. 1, 2014, the entire contents of which is hereby incorporated by reference.

### SEQUENCE LISTING

The instant application contains a Sequence Listing which has been submitted electronically in ASCII format and is hereby incorporated by reference in its entirety. Said ASCII copy, created on Apr. 1, 2015, is named 123436-18202_SL.txt and is 40,950 bytes in size.

### BACKGROUND OF THE INVENTION

Various tissue-derived products are used to regenerate, repair, or otherwise treat diseased or damaged tissues and organs. Such products can include intact tissue grafts and/or acellular or reconstituted acellular tissues (e.g., acellular tissue matrices from skin, intestine, or other tissues, with or without cell seeding), which may be derived from a donor of a different species from the recipient (xenograft) or from a donor of the same species as the recipient (allograft). For example, a collagen-containing material may be made from porcine tissue and implanted in a human patient.

In recipient animals (e.g., humans) that do not express the enzyme β-D-galactosyl-1,4-N-acetyl-D-glucosaminide α-1,3 galactosyl-transferase (α-1,3 galactosyltransferase; “αGT”), which catalyzes the binding of α-1,3galactose (Gal) on N-acetyllactosamine (Galβ1,4GlcNAc) to produce terminal Galα1,3Galβ1,4GlcNAc-R (“galactose-alpha-1,3-galactose,” “α-gal epitope,” or “α-gal”) on an acceptor substrate, a major problem of xenotransplanted tissue is hyperacute rejection of xenografts in such recipients that is largely, if not exclusively, due to the action of antibodies specific for the α-gal epitope on the surface of cells in the xenograft.

The α-gal epitope is expressed in non-primate mammals and in New World monkeys (monkeys of South America) as well as on macromolecules such as proteoglycans of the extracellular components, but is absent in Old World primates (monkeys of Asia and Africa and apes) and humans (U. Galili et al. (1988) J. Biol. Chem. 263: 17755). Anti-gal antibodies are produced in humans and primates as a result of an immune response to α-gal epitope carbohydrate structures on gastrointestinal bacteria. U. Galili et al. (1988) Infect. Immun. 56:1730; R. M. Hamadeh et al. (1992) J. Clin. Invest. 89:1223.

Since non-primate mammals (e.g., pigs) produce α-gal epitopes, xenotransplantation of tissue material from these mammals into primates often results in rejection because of primate anti-Gal antibody binding to these epitopes. The binding results in the destruction of the tissue material by complement fixation and antibody dependent cell cytotoxicity. U. Galili et al. (1993) Immunology Today 14:480; M. Sandrin et al. (1993) Proc. Natl. Acad. Sci. USA 90:11391; H. Good et al. (1992) Transplant. Proc. 24:559; B. H. Collins et al. (1995) J. Immunol. 154: 5500. Accordingly, when animals that produce α-gal epitopes are used as the tissue source for treatment of diseased or damaged tissue or organs, the removal of α-gal epitopes from cells and from extracellular components of the tissue material can diminish the immune response associated with anti-gal antibody binding to α-gal epitopes.

Enzymes such as alpha-galactosidases from green coffee beans and Bacteroides, which substantially eliminate α-gal epitopes from cells and from extracellular components of a collagen-containing material, have been identified and used to prepare tissue products (see, e.g., Luo, et al. (1999) Xenotransplantation 6(4):238-48; U.S. Pat. No. 7,951,552). However, supply issues have decreased the availability of these enzymes, thereby increasing the cost and availability of tissue products to treat subjects in need thereof. Accordingly, there is a need for additional enzymes and methods for treating tissue products to reduce or control the immune response of the tissue products upon implantation.

In one aspect, the present disclosure provides methods for preparation of a non-human tissue matrix for xenotransplantation. The methods can include contacting a collagen-containing tissue matrix with an isolated Trichoderma reesei or Clostridium cellulyticum alpha-galactosidase in an amount and for a time sufficient to remove an α-gal epitope from the tissue matrix, thereby preparing the non-human tissue matrix for xenotransplantation.

In one embodiment, the alpha-galactosidase comprises an amino acid sequence having at least 85% identity to the entire amino acid sequence set forth in a sequence selected from the group consisting of SEQ ID NOs:2, 4, 6, and 8. In another embodiment, the alpha-galactosidase is encoded by a nucleic acid molecule comprising a nucleotide sequence having at least 85% identity to the entire nucleotide sequence set forth in a sequence selected from the group consisting of SEQ ID NOs:1, 3, 5, and 7. In another embodiment, the alpha-galactosidase is encoded by a nucleic acid molecule, said nucleic acid molecule encoding a protein comprising an amino acid sequence having at least 85% identity to the entire amino acid sequence set forth in a sequence selected from the group consisting of SEQ ID NOs:2, 4, 6, and 8.

In one embodiment, the tissue matrix is an acellular tissue matrix. In another embodiment, the tissue matrix comprises a dermal tissue matrix.

In one embodiment, the tissue matrix is obtained from a tissue selected from fascia, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, and intestinal tissue.

In one embodiment, the methods of the invention further comprise treating the tissue matrix to remove at least some of the cells and cellular components, e.g., substantially all the cells and cellular components, from the tissue matrix.

In one embodiment, the methods of the invention further comprise treating the tissue matrix to remove substantially all cells and cellular components prior to contacting the tissue matrix with the alpha-galactosidase.

In one embodiment, the at least one collagen-containing tissue matrix includes two or more tissue matrices.

In one embodiment, the methods of the invention further comprise packaging the tissue matrix.

In one embodiment, the methods of the invention further comprise sterilizing the tissue matrix.

In another aspect, the present invention provides a tissue matrix prepared according to the method of the invention.

### BRIEF DESCRIPTION OF THE DRAWINGS

FIGS. 1A-1J show acellular tissue matrices stained for the α-gal epitope. FIGS. 1C-1E and 1H-1J show acellular tissue matrices stained for the α-gal epitope following treatment with the indicated enzymes using the methods described in Example 1. FIGS. 1A and 1F show untreated control acellular tissue matrices consisting of porcine dermis that was not exposed to enzyme and stained for the α-gal epitope, and FIGS. 1B and 1G are positive controls consisting of porcine dermis treated with green coffee bean alpha-galactosidase. The presence of α-gal epitope is indicated by a dark gray color.

### DESCRIPTION OF EXEMPLARY EMBODIMENTS

The present invention is based, at least in part, on the discovery of microbial alpha-galactosidases that remove terminal alpha-1,3 linked galactose residues from animal tissue and methods of using such alpha-galactosidases for preparing a non-human tissue matrix that may be used, e.g., as a tissue product. As described in the appended examples below, it has been discovered that of the multitude of microbial alpha-galactosidases that have been identified, only alpha-glactosidases from Trichoderma reesei and Clostridium cellulolyticum effectively cleave α-gal epitopes from non-human tissue and, thus, e.g., reduce the immunogenicity of tissue matrices prepared for xenotransplantation.

Accordingly, the present disclosure provides methods for preparing non-human tissue or tissue matrices for implantation into human patients.

### I. DEFINITIONS

Definitions of certain terms are first defined below. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, the term “subject” refers to human and non-human animals, e.g., veterinary subjects. The term “non-human animal” includes all vertebrates, e.g., mammals and non-mammals, such as non-human primates, mice, rabbits, sheep, dog, cat, horse, cow, chickens, amphibians, and reptiles. In one embodiment, the subject is a human.

As used herein “tissue product” refers to any tissue that contains extracellular matrix proteins. Various tissues can be used to produce tissue products for, e.g., treating a subject in need thereof. For example, various tissue products for regeneration, repair, augmentation, reinforcement, and/or treatment of human tissues that have been damaged or lost due to various diseases and/or structural damage (e.g., from trauma, surgery, atrophy, and/or long-term wear and degeneration) have been produced. Such products can include, for example, acellular or partially decellularized tissue matrices, decellularized tissue matrices that have been repopulated with exogenous cells, and/or cellular tissues.

The tissues can be selected from a variety of tissue sources including skin (dermis or whole skin), fascia, pericardial tissue, dura, umbilical cord tissue, placental tissue, cardiac valve tissue, ligament tissue, adipose tissue, tendon tissue, arterial tissue, venous tissue, neural connective tissue, urinary bladder tissue, ureter tissue, and intestinal tissue. The methods described herein can be used to process any collagenous tissue type, and for any tissue matrix product. For example, a number of biological scaffold materials are described by Badylak et al. (Acta Biomaterialia (2008), doi:10.1016/j.actbio.2008.09.013), and the methods of the present disclosure can be used to treat those or other tissue products known in the art.

In some cases, the tissue products can be provided as decellularized tissue matrices. Suitable acellular tissue matrices are described further below. In some cases, the methods of the may further include processing intact tissue to remove cells or other materials. The tissues can be completely or partially decellularized to yield acellular tissue matrices or extracellular tissue materials. For example, various tissues, such as skin, intestine, bone, cartilage, adipose tissue, nerve tissue (e.g., nerve fibers or dura), tendons, ligaments, or other tissues can be completely or partially decellularized to produce tissue products useful for patients. In some cases, these decellularized products can be used without addition of exogenous cellular materials (e.g., stem cells). In certain cases, these decellularized products can be seeded with cells from autologous sources or other sources to facilitate treatment. Suitable processes for producing acellular tissue matrices are described below.

### II. METHODS OF PREPARING TISSUE PRODUCTS

The present disclosure provides methods for preparation of a non-human tissue matrix for xenotransplantation. The methods can include contacting a collagen-containing tissue matrix with an isolated Trichoderma reesei or Clostridium cellulyticum alpha-galactosidase in an amount and for a time sufficient to remove an α-gal epitope from the tissue matrix, thereby preparing the non-human tissue matrix for xenotransplantation. A “sufficient amount and time to remove an α-gal epitope from the tissue matrix” is the amount of the enzyme and the time that the tissue matrix is contacted with the enzyme to reduce the immunogenicity of the tissue matrix prepared for xenotransplantation. An immune response can be measured using a number of immunoassays, including monocyte activation assays, phagocytosis assays, and/or oxidative burst assays, which are readily known to one of ordinary skill in the art.

The term “reduce” with respect to the immunogenicity of a tissue matrix refers to a statistically significant decrease in such level. The decrease can be, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more.

Any suitable enzyme concentration, buffer, pH, temperature, and treatment time can be used as long as it is sufficient to remove an α-gal epitope from the tissue matrix. In some embodiments, the conditions suitable for treating a tissue sample as described herein may include about 50 U/L to about 400 U/L of an α-galactosidase in a buffer at a physiologically acceptable pH (e.g., about pH 6.0 to about pH 8.0) and temperature (e.g., about 20° C. to about 40° C.), e.g., about 50, 60, 70, 80, 90, 100, 110, 120, 130, 140, 150, 160, 170, 180, 190, 200, 210, 220, 230 240, 250, 260, 270, 280, 290, 300, 310, 320, 330, 340, 350, 360, 370, 380, 390, or about 400 U/L an α-galactosidase in a, e.g., phosphate buffer, having a pH of about 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or about 8.0, at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C. Ranges and values intermediate to the above recited ranges and values are also contemplated to be part of the invention. In an exemplary embodiment, the tissue sample is treated with an α-galactosidase at a concentration of 300 U/L prepared in 100 mM phosphate buffer at pH 6.0. In other embodiments, the concentration of α-galactosidase is increased to 400 U/L for adequate removal of the α-gal epitopes from the harvested tissue.

Species that can serve as recipients of a tissue matrix and donors of tissues or organs for the production of the tissue matrix include, without limitation, mammals, such as humans and non-human primates (e.g., monkeys, baboons, or chimpanzees).

In one embodiment, the methods further include at least partial decellularization of the tissue. The decellularization step may be performed before contacting the tissue with the glactosidase or concomitantly with contacting the tissue with the galactosidase. The tissue may be a dermal tissue.

The present disclosure also provides methods of treating a subject with a tissue matrix of the present invention. The methods can include preparing a tissue matrix as described herein, identifying a mammalian subject as having an organ, or tissue, in need of repair or amelioration; and placing the tissue matrix in or on the organ or tissue. In one embodiment, the subject is human. The methods can further comprise administration to the subject of one or more agents, e.g., a cell growth factor, an angiogenic factor, a differentiation factor, a cytokine, a hormone, or a chemokine. The one or more agents can be in the tissue matrix placed in the subject or they can be injected or infused into the subject separately from the tissue matrix. The organ or tissue of the subject can be, without limitation, skin, bone, cartilage, meniscus, dermis, myocardium, periosteum, artery, vein, stomach, small intestine, large intestine, diaphragm, tendon, ligament, neural tissue, striated muscle, smooth muscle, bladder, urethra, ureter, gingival, or fascia (e.g., abdominal wall fascia).

As used herein, the terms “treating” or “treatment” refer to a beneficial or desired result including, but not limited to, alleviation or amelioration of one or more symptoms. “Treatment” can also mean prolonging survival as compared to expected survival in the absence of treatment.

As used herein, the term “placing” a tissue matrix includes, without limitation, setting, injecting, infusing, pouring, packing, layering, spraying, and encasing the composition. In addition, placing “on” a recipient tissue or organ means placing in contact with the recipient tissue or organ.

Suitable T. reesei and C. cellulolyticum alpha-galactosidases for use in the methods of the present invention may be either naturally occurring (native) or genetically engineered. For example, suitable enzymes may be obtained by, for example, fermentation of the organisms and use of an appropriate purification scheme using standard protein purification techniques. For example, cell supernatants may be collected and concentrated (e.g., by ultrafiltration) and applied to, for example, an isoelectric focusing matrix to identify fractions having alpha-galactosidase activity. Active fractions may be further purified using, for example, ion-exchange chromatography and/or gel filtration. Alternatively, recombinant DNA techniques may be used to produce a T. reesei and C. cellulolyticum alpha-galactosidase comprising the whole or a segment of the protein (a functional fragment of the protein). For example, recombinant DNA techniques may be used to clone a nucleotide sequence encoding a segment or the whole protein into a vector (such as an expression vector) and transform a cell for production of the protein. A T. reesei and C. cellulolyticum alpha-galactosidase comprising the whole or a segment of the protein may also be synthesized chemically using standard peptide synthesis techniques.

The nucleotide and amino acid sequences of T. reesei and C. cellulolyticum alpha-galactosidases may be found in, for example, GenBank (see, e.g., www.ncbi.nlm.nih.gov) or UniProt (see, e.g., www.uniprot.org/uniprot). In particular, T. reesei has three genes (aglI, aglII, and aglIII) that encode proteins having alpha-galactosidase activity for use in the methods of the invention, the nucleotide and amino acid sequence of which may be found in, for example, GenBank Accession numbers GI:1580815 (SEQ ID NO:1); GI:74630547 (SEQ ID NO:2); GI:1580817 (SEQ ID NO:3); GI:74630548 (SEQ ID NO:4); GI:1580811 (SEQ ID NO:5); and GI:74630544 (SEQ ID NO:6). It should be understood that any one, two or three of the T. reesei nucleic acid molecules or proteins may be used in the methods of the invention. C. cellulolyticum has a single alpha-galactosidase gene encoding a protein having alpha-galactosidase activity for use in the methods of the invention, the nucleotide and amino acid sequence of which may be found in, for example, GenBank Accession numbers GI:110588919 (nucleotides 3121-4935 of SEQ ID NO:7); and GI:219998992 (SEQ ID NO:8). The entire contents of each or the foregoing GenBank records are incorporated herein by reference.

As used herein, the terms an “isolated molecule”, such as an “isolated nucleic acid molecule”, “an isolated polypeptide”, “an isolated protein”, is one which is separated from other molecules which are present in the natural source of the molecule. In one embodiment, an “isolated nucleic acid molecule”, is free of sequences (such as protein-encoding sequences) which naturally flank the nucleic acid (i.e., sequences located at the 5′ and 3′ ends of the nucleic acid) in the genomic DNA of the organism from which the nucleic acid is derived. For example, in various embodiments, an isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. In another embodiment, an “isolated” molecule can be substantially free of other cellular material, or culture medium when produced by recombinant techniques, or substantially free of chemical precursors or other chemicals when chemically synthesized. A molecule that is substantially free of cellular material includes preparations having less than about 30%, 20%, 19%, 18%, 17%, 16%, 15%, 14%, 13%, 12%, 11%, 10%, 9%, 8%, 7%, 6%, or about 5% of heterologous molecules and which retains alpha-galactosidase activity.

RNA or DNA encoding the alpha-galactosidases may be readily isolated, amplified, and/or sequenced using conventional procedures (e.g., by using oligonucleotide probes that are capable of binding specifically to the relevant genes, as described in, for example, Innis et al. in PCR Protocols. A Guide to Methods and Applications, Academic (1990), and Sanger et al., Proc Natl Acad Sci USA 74:5463 (1977)). A nucleic acid molecule so amplified may be cloned into an appropriate vector and characterized by DNA sequence analysis. Furthermore, nucleotides corresponding to all or a portion of an isolated nucleic acid molecule for use in the methods of the invention can be prepared by standard synthetic techniques, e.g., using an automated DNA synthesizer.

In one embodiment, an isolated nucleic acid molecule for use in the methods of the invention comprises a nucleic acid molecule which has a nucleotide sequence complementary to the nucleotide sequence of a nucleic acid molecule encoding a T. reesei or a C. cellulolyticum alpha-galactosidase. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule for use in the methods of the invention can comprise only a portion of a nucleic acid sequence which encodes a T. reesei or a C. cellulolyticum alpha-galactosidase. Such nucleic acid molecules can be used, for example, as a probe or primer. The probe/primer typically is used as one or more substantially purified oligonucleotides. The oligonucleotide typically comprises a region of nucleotide sequence that hybridizes under stringent conditions to at least about 7, preferably about 15, more preferably about 25, 50, 75, 100, 125, 150, 175, 200, 250, 300, 350, or 400 or more consecutive nucleotides of a nucleic acid molecule for use in the methods of the invention.

The invention further encompasses nucleic acid molecules that differ, due to degeneracy of the genetic code, from the nucleotide sequence of nucleic acid molecules encoding a T. reesei or a C. cellulolyticum alpha-galactosidase protein and thus encode the same protein. It will be appreciated by those skilled in the art that DNA sequence polymorphisms that lead to changes in the amino acid sequence can exist within a population. Such genetic polymorphisms can exist among individuals within a population due to natural allelic variation. An allele is one of a group of genes which occur alternatively at a given genetic locus. In addition, it will be appreciated that DNA polymorphisms that affect RNA expression levels can also exist that may affect the overall expression level of that gene (e.g., by affecting regulation or degradation).

Accordingly, in one embodiment a nucleic acid molecule suitable for use in the methods of the invention is at least about 40% identical, about 50%, 60%, 70%, 80%, 81%, 82%, 83%, 84%, 85%, 86%, 87%, 88%, 89%, 90%, 91%, 92%, 93%, 94%, 95%, 96%, 97%, 98%, or about 99% identical to the nucleotide sequence of a T. reesei or a C. cellulolyticum alpha-galactosidase.

In addition to naturally occurring allelic variants of a nucleic acid molecule of the invention that can exist in the population, the skilled artisan will further appreciate that sequence changes can be introduced by mutation thereby leading to changes in the amino acid sequence of the encoded protein, without altering the biological activity of the protein encoded thereby. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the homologs of various species may be essential for activity and thus would not be likely targets for alteration.

Accordingly, another aspect of the invention pertains to nucleic acid molecules encoding a variant protein that contain changes in amino acid residues that are not essential for activity. Such variant proteins differ in amino acid sequence from the naturally-occurring proteins, yet retain biological activity. In one embodiment, such a variant protein has an amino acid sequence that is at least about 40% identical, 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of a T. reesei or a C. cellulolyticum alpha-galactosidase.

Identity or similarity with respect to parent amino acid sequence is defined herein as the percentage of amino acid residues in the candidate sequence that are identical (i.e., same residue) or similar (i.e., amino acid residue from the same group based on common side-chain properties, supra) with the parent molecule residues, after aligning the sequences and introducing gaps, if necessary, to achieve the maximum percent sequence identity.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length.

The determination of percent identity between two sequences can be accomplished using a mathematical algorithm (see, e.g., Karlin and Altschul (1990) Proc. Natl. Acad. Sci. USA 87:2264-2268; Karlin and Altschul (1993) Proc. Natl. Acad. Sci. USA 90:5873-5877; Altschul, et al. (1990) J. Mol. Biol. 215:403-410; Altschul et al. (1997) Nucleic Acids Res. 25:3389-3402; Myers and Miller, (1988) CABIOS 4:11-17; Pearson and Lipman (1988) Proc. Natl. Acad. Sci. USA 85:2444-2448).

An isolated nucleic acid molecule encoding a variant protein can be created by introducing one or more nucleotide substitutions, additions or deletions into the nucleotide sequence of nucleic acids, such that one or more amino acid residue substitutions, additions, or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

Biologically active portions of a T. reesei or a C. cellulolyticum alpha-galactosidase are also included within the scope of the present invention. Such biologically active portions include polypeptides comprising amino acid sequences sufficiently identical to or derived from the amino acid sequence of a T. reesei or a C. cellulolyticum alpha-galactosidase protein, which include fewer amino acids than the full length protein, and exhibit at least one activity of the corresponding full-length protein. Typically, biologically active portions comprise a domain or motif with at least one activity of the corresponding full-length protein. A biologically active portion of a protein for use in the methods of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of the protein (e.g., removal of α-gal epitopes).

The invention also provides chimeric or fusion proteins comprising a T. reesei or a C. cellulolyticum alpha-galactosidase protein or a segment thereof. As used herein, a “chimeric protein” or “fusion protein” comprises all or part (preferably a biologically active part) of a T. reesei or a C. cellulolyticum alpha-galactosidase protein operably linked to a heterologous polypeptide (i.e., a polypeptide other than the alpha-galactosidase protein). Within the fusion protein, the term “operably linked” is intended to indicate that the alpha-galactosidase protein or segment thereof and the heterologous polypeptide are fused in-frame to each other. The heterologous polypeptide can be fused to the amino-terminus or the carboxyl-terminus of the alpha-galactosidase protein or segment.

Chimeric and fusion proteins of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide for use in the methods of the invention.

A signal sequence can be used to facilitate secretion and isolation of T. reesei or a C. cellulolyticum alpha-galactosidase proteins. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to T. reesei or a C. cellulolyticum alpha-galactosidase proteins, fusion proteins or segments thereof having a signal sequence, as well as to such proteins from which the signal sequence has been proteolytically cleaved (i.e., the cleavage products). In one embodiment, a nucleic acid sequence encoding a signal sequence can be operably linked in an expression vector to a nucleitc acid molecule encoding a protein of interest, such as a T. reesei or a C. cellulolyticum alpha-galactosidase protein, or a segment thereof. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a poly-histidine tag, a strep-tag, a FLAG-tag, a GST domain, etc.

Nucleic acid molecules encoding the polypeptides, or functional fragments thereof, for use in the methods of the invention may be incorporated in suitable recombinant vectors. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, namely expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant vectors of the invention can comprise a nucleic acid encoding a polypeptide in a form suitable for expression of the nucleic acid in a host cell. In some embodiments, this means that the recombinant vectors may include one or more regulatory sequences, selected on the basis of the host cells to be used for expression, which is operably linked to the nucleic acid sequence to be expressed (i.e., a recombinant expression vector). Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Methods in Enzymology: Gene Expression Technology vol. 185, Academic Press, San Diego, Calif. (1991). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like.

The expression vectors of the invention can be introduced into host cells to thereby produce proteins or peptides, including fusion proteins or peptides, encoded by nucleic acids as described herein.

The recombinant expression vectors of the invention can be designed for expression of a polypeptide, or functional fragment thereof, in prokaryotic (e.g., E. coli) or eukaryotic cells (e.g., insect cells {using baculovirus expression vectors}, yeast cells or mammalian cells). Suitable host cells are discussed further in Goeddel, supra, and include, for example, E. coli cells, Bacillus cells, Saccharomyces cells, Pochia cells, NS0 cells, COS cells, Chinese hamster ovary (CHO) cells or myeloma cells. The RNA or DNA also may be modified, for example, by substituting bases to optimize for codon usage in a particular host or by covalently joining to the coding sequence of a heterologous polypeptide. Such an approach would be the basis for developing a subunit vaccine. Alternatively, the recombinant expression vector can be transcribed and translated in vitro.

Another aspect of the invention pertains to host cells into which a recombinant vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

### III. ACELLULAR TISSUE MATRICES

The term “acellular tissue matrix,” as used herein, refers generally to any tissue matrix that is substantially free of cells and/or cellular components. Skin, parts of skin (e.g., dermis), and other tissues such as blood vessels, heart valves, fascia, cartilage, adipose tissue, bone, and nerve connective tissue may be used to create acellular matrices within the scope of the present disclosure. Acellular tissue matrices can be tested or evaluated to determine if they are substantially free of cell and/or cellular components in a number of ways. For example, processed tissues can be inspected with light microscopy to determine if cells (live or dead) and/or cellular components remain. In addition, certain assays can be used to identify the presence of cells or cellular components. For example, DNA or other nucleic acid assays can be used to quantify remaining nuclear materials within the tissue matrices. Generally, the absence of remaining DNA or other nucleic acids will be indicative of complete decellularization (i.e., removal of cells and/or cellular components). Finally, other methods which identify cell-specific components (e.g., surface antigens) can be used to determine if the tissue matrices are acellular. Skin, parts of skin (e.g., dermis), and other tissues such as blood vessels, heart valves, fascia, cartilage, bone, and nerve connective tissue may be used to create acellular matrices within the scope of the present disclosure.

In general, the steps involved in the production of an acellular tissue matrix include harvesting the tissue from a donor (e.g., an animal source) and cell removal under conditions that preserve biological and structural function. In certain embodiments, the process includes chemical treatment to stabilize the tissue and avoid biochemical and structural degradation together with or before cell removal. In various embodiments, the stabilizing solution arrests and prevents osmotic, hypoxic, autolytic, and proteolytic degradation, protects against microbial contamination, and reduces mechanical damage that can occur with tissues that contain, for example, smooth muscle components (e.g., blood vessels). The stabilizing solution may contain an appropriate buffer, one or more antioxidants, one or more oncotic agents, one or more antibiotics, one or more protease inhibitors, and/or one or more smooth muscle relaxants.

The tissue is then placed in a decellularization solution to remove viable cells (e.g., epithelial cells, endothelial cells, smooth muscle cells, and fibroblasts) from the structural matrix without damaging the biological and structural integrity of the collagen matrix. The decellularization solution may contain an appropriate buffer, salt, an antibiotic, one or more detergents (e.g., TRITON X100™, sodium deoxycholate, polyoxyethylene (20) sorbitan mono-oleate), one or more agents to prevent cross-linking, one or more protease inhibitors, and/or one or more enzymes. In some embodiments, the decellularization solution comprises 1% TRITON X-100™ in RPMI media with Gentamicin and 25 mM EDTA (ethylenediaminetetraacetic acid). In some embodiments, the tissue is incubated in the decellularization solution overnight at 37° C. with gentle shaking at 90 rpm. In certain embodiments, additional detergents may be used to remove fat from the tissue sample. For example, in some embodiments, 2% sodium deoxycholate is added to the decellularization solution.

After the decellularization process, the tissue sample is washed thoroughly with saline. In some exemplary embodiments, the decellularized tissue is then treated overnight at room temperature with a deoxyribonuclease (DNase) solution. In some embodiments, the tissue sample is treated with a DNase solution prepared in DNase buffer (20 mM HEPES (4-(2-hydroxyethyl)-1-piperazineethanesulfonic acid), 20 mM CaCl₂ and 20 mM MgCl₂). Optionally, an antibiotic solution (e.g., Gentamicin) may be added to the DNase solution. Any suitable buffer can be used as long as the buffer provides suitable DNase activity.

After washing the tissue thoroughly with saline to remove the DNase solution, the tissue sample is contacted with a T. reesei or a C. cellulolyticum alpha-galactosidase.

After treatment with an alpha-galactosidase, an assay may be performed to determine if the collagen-containing tissue matrix has been altered such that a human immune response is reduced. A number of suitable assays may be performed. For example, suitable assays can include monocyte activation assays, phagocytosis assays, and oxidative burst assays.

In some embodiments, the assay may be performed on a segment or portion of the processed tissue, and other portions of the tissue may be used in subsequent medical or surgical procedures. In other embodiments, the assay may be performed on one or more samples from a batch of multiple samples, and samples not subjected to the assay may be subsequently selected for use in treating a patient.

In some embodiments, after the acellular tissue matrix is formed, histocompatible, viable cells are seeded in the acellular tissue matrix to produce a graft that may be further remodeled by the host. In some embodiments, histocompatible viable cells may be added to the matrices by standard in vitro cell co-culturing techniques prior to transplantation, or by in vivo repopulation following transplantation. In vivo repopulation can be by the recipient's own cells migrating into the acellular tissue matrix or by infusing or injecting cells obtained from the recipient or histocompatible cells from another donor into the acellular tissue matrix in situ. Various cell types can be used, including embryonic stem cells, adult stem cells (e.g. mesenchymal stem cells), and/or neuronal cells. In various embodiments, the cells can be directly applied to the inner portion of the acellular tissue matrix just before or after implantation. In certain embodiments, the cells can be placed within the acellular tissue matrix to be implanted, and cultured prior to implantation.

The present invention is further illustrated by the following examples, which should not be construed as further limiting. The contents of all figures and all references, patents and published patent applications cited throughout this application, as well as the Figures, are expressly incorporated herein by reference in their entirety.

### Example 1

### Identification of Microbial Alpha-Galactosidases for Removal of Terminal Alpha-1,3-Linked Galactose Residues from Tissue

Publically available databases were surveyed to identify commercially available alpha-galactosidases and microbial organisms that produce alpha-galactosidases.

Enzymes were purchased or partially purified and tested for alpha-glactosidase activity on p-nitrophenyl alpha-galatctosidase (pNGP) substrate. Briefly, samples were diluted in either phosphate buffer pH 6.0 or 20 mM HEPES, 1.2 M NaCl, 20 mM CaCl2, 20 mM MgCl2, pH 7.4. pNGP was then added to a final concentration of 1 mM and the samples incubated at 37 C. Enzyme activity was assessed by measuring resulting absorbance at 405 nm.

Those microbial enzymes having activity on pNGP were subsequently tested for their ability to reduce and/or eliminate α-gal epitopes on tissue. The enzymes and the source of the alpha-galactosidase tested are listed below.
Aspergillus niger (partially purified materials were purchased from MarCor and Specialty Enzyme; tissue samples were tested with concentrations up to 20,000 U/L)
   AglA
   AglB
   AglC (purified recombinant AglC was purchased from Megazyme; tested on tissue at concentrations up to 100,000 U/L)
   Agl Unknown
Trichoderma reesei (purified material from MPBio having activity on tissue at 400 U/L, and crude and partially purified (Q-HiTrap) from crude culture supernatants)
   AglI
   AglII
   AglIII
Guar (purified recombinant (E. coli) form purchased from Megazyme; tested on tissue at concentrations up to 100,000 U/L)
Phaseus
Cellvibrio mixtus (recombinant (E. coli) form purchased from Prozomix; tested on tissue at concentrations up to 20,000 U/L)
Clostridium cellulyticum (recombinant (E. coli) form purchased from Prozomix; active on tissue at 4,000 U/L)
Saccharomyces cerevisiae (recombinant form purchased from CUSABIO and a proprietary source; tested on tissue at concentrations up to 20,000 U/L, or 50 mg/ml)
Bacillus subtilis (recombinant (E. coli) form purchased from Genway Biologics)
Xanthomonas manihotis (recombinant (E. coli) form purchased from New England BioLabs; tested on tissue at concentrations up to 50,000 U/L)
Escherechia coli (recombinant form purchased from U.S. biological; tested on tissue at concentrations up to 13 mg/ml)

Porcine skin was collected from an abattoir and split to 1.3 mm by physically removing the epidermis and subcutaneous fat. The remaining dermal tissue was de-contaminated with peracetic acid. Following de-contamination, the tissue was processed under aseptic conditions. The dermal tissue was decellularized for 24 hours with detergents to remove viable cells. Cellular debris and residual chemicals are removed by washing in PBS. The resulting porcine acellular dermal matrix (pADM) was stored at 4° C. until use.

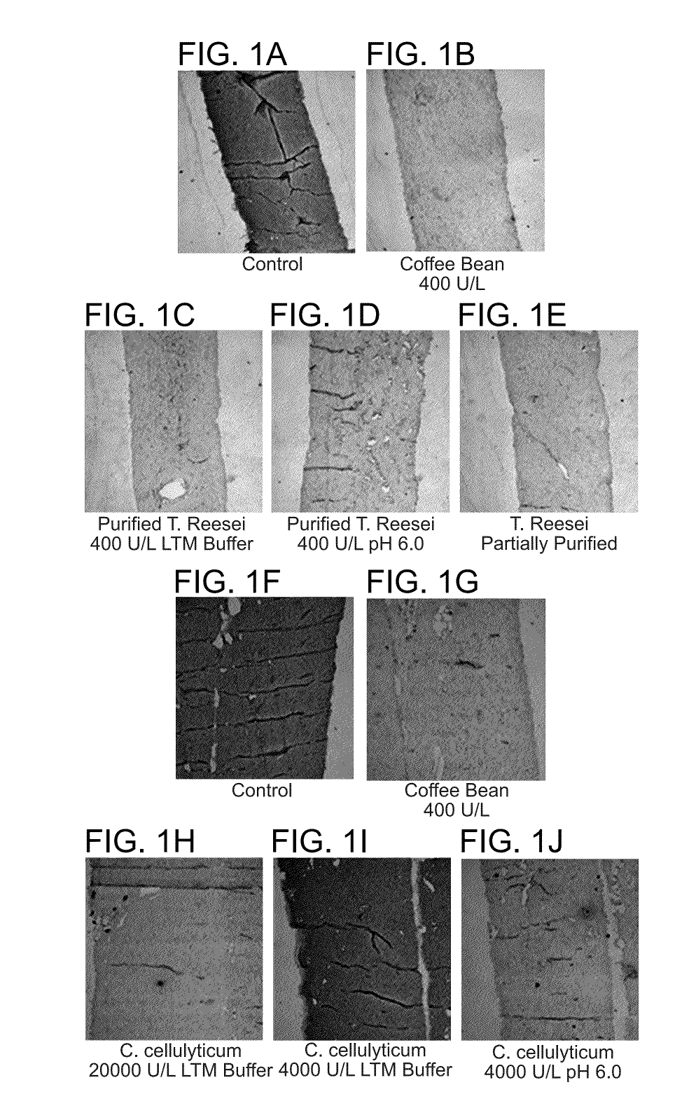

Enzyme preparations from those microbes identified as having activity on pNGP were tested on porcine dermis in a phosphate buffer, pH 6.0 or LTM DNase buffer, pH 7.4 (20 mM HEPES, 1.2 M NaCl, 20 mM CaCl2, 20 mM MgCl2) for up to 24 hours. Histological sections were prepared and stained with the IB4 lectin which specifically recognizes the α-gal epitope. Of the multitude of microbial enzymes annotated and/or tested as having alpha-galactosidase activity on pNGP, only two alpha-galactosidases from T. reesei or a C. cellulolyticum were effective at removing the α-gal epitopes from tissues. Specifically, the tissue sections treated with partially purified and commercially available alpha-galactosidase from T. reesei were as effective as the coffee bean alpha-galactosidase at reducing staining specific for the α-gal epitopes on tissue (see, e.g., FIGS. 1B-1E). In addition, recombinant C. cellulolyticum alpha-galactosidase was also as effective as the coffee bean alpha-galactosidase at reducing staining specific for the α-gal epitopes on tissue but demonstrated greater activity in reduced pH buffers (see, e.g., FIGS. 1G-1J).

### EQUIVALENTS

In describing embodiments of the invention, specific terminology is used for the sake of clarity. For purposes of description, each specific term is intended to at least include all technical and functional equivalents that operate in a similar manner to accomplish a similar purpose. Additionally, in some instances where a particular embodiment of the invention includes a plurality of system elements or method steps, those elements or steps may be replaced with a single element or step; likewise, a single element or step may be replaced with a plurality of elements or steps that serve the same purpose. Further, where parameters for various properties are specified herein for embodiments of the invention, those parameters can be adjusted up or down by 1/20th, 1/10th, ⅕th, ⅓rd, ½, etc., or by rounded-off approximations thereof, unless otherwise specified. Moreover, while this invention has been shown and described with references to particular embodiments thereof, those skilled in the art will understand that various substitutions and alterations in form and details may be made therein without departing from the scope of the invention; further still, other aspects, functions and advantages are also within the scope of the invention. The contents of all references, including patents and patent applications, cited throughout this application are hereby incorporated by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

We claim:

1. A method for preparing a non-human tissue matrix for transplantation, comprising:
contacting a collagen-containing tissue matrix with an isolated Trichoderma reesei or Clostridium cellulyticum alpha-galactosidase in an amount and for a time sufficient to remove an α-gal epitope from the tissue matrix, thereby preparing the tissue matrix for transplantation into a human patient.

2. The method of claim 1, wherein the alpha-galactosidase comprises an amino acid sequence having at least 85% identity to the entire amino acid sequence set forth in a sequence selected from the group consisting of SEQ ID NOs:2, 4, 6, and 8.

3. The method of claim 1, wherein the alpha-galactosidase is encoded by a nucleic acid molecule comprising a nucleotide sequence having at least 85% identity to the entire nucleotide sequence set forth in a sequence selected from the group consisting of SEQ ID NOs:1, 3, 5, and 7.

4. The method of claim 1, wherein the alpha-galactosidase is encoded by a nucleic acid molecule, said nucleic acid molecule encoding a protein comprising an amino acid sequence having at least 85% identity to the entire amino acid sequence set forth in a sequence selected from the group consisting of SEQ ID NOs:2, 4, 6, and 8.

5. The method of claim 1, wherein the tissue matrix is an acellular tissue matrix.

6. The method of claim 1, wherein the tissue matrix comprises a dermal tissue matrix.

7. The method of claim 1, wherein the tissue matrix is obtained from a tissue selected from fascia, pericardial tissue, dura, umbilical cord tissue, placental tissue, cardiac valve tissue, ligament tissue, tendon tissue, arterial tissue, venous tissue, neural connective tissue, urinary bladder tissue, ureter tissue, and intestinal tissue.

8. The method of claim 1, further comprising treating the tissue matrix to remove at least some of the cells and cellular components from the tissue matrix.

9. The method of claim 8, wherein the tissue matrix is treated to remove substantially all cells and cellular components prior to contacting the tissue matrix with the alpha-galactosidase.

10. The method of claim 8, wherein substantially all the cells and cellular components are removed from the tissue matrix.

11. The method of claim 1, wherein the at least one collagen-containing tissue matrix includes two or more tissue matrices.

12. The method of claim 1, further comprising packaging the tissue matrix.

13. The method of claim 1, further comprising sterilizing the tissue matrix.

14. A tissue matrix prepared according to the method of claim 1.
